(19) United States
(12) Patent Application Publication
(10) Pub. No.: US 2010/0144682 A1
(43) Pub. Date: Jun. 10, 2010

(54) COMPOSITION BASED ON SALICYLIC ACID DERIVATIVES

(75) Inventors: Anne-Laure Bernard, Neuilly / Seine (FR); Marine Bouvier, Yerres (FR)

Correspondence Address: OBLON, SPIVAK, MCCLELLAND MAIER & NEUSTADT, L.L.P., 1940 DUKE STREET, ALEXANDRIA, VA, 22314, US

(73) Assignee: L'OREAL, Paris (FR)

(21) Appl. No.: 12/525,586

(22) PCT Filed: Jan. 31, 2008
(86) PCT No.: PCT/EP2008/051213

§ 371 Date: Dec. 30, 2009

Related U.S. Application Data
(60) Provisional application No. 60/894,257, filed on Mar. 12, 2007.

(30) Foreign Application Priority Data
Feb. 1, 2007 (FR) 0752995

(51) Int. Cl. A61K 8/36; A61Q 19/10

(52) U.S. Cl. 514/162; 510/159

(57) ABSTRACT

The present invention relates to an aqueous composition for topical application comprising at least one (C⁸-C¹⁴)alkyl betaine and at least one salicylic acid acylated derivative, the molar ratio of the (C⁸-C¹⁴)alkyl betaine(s) to the salicylic acid derivative(s) being equal to or greater than 1. The composition can be used for peeling or cleaning the skin or also for treating greasy skin.

The present invention relates to a composition based on a salicylic acid derivative and on an alkyl betaine and to the use of this composition in the care of the skin and in particular for peeling or cleaning human skin or for the treatment of greasy skin.

Peels are a well known means for improving the surface appearance of the skin, in particular for treating irregular visible and/or tactile features of human skin, and for example toning down defects of pigmentation, such as actinic lentigines or signs of acne or chicken pox, or for smoothing unevennesses in the texture of the skin, in particular wrinkles and fine lines.

These peels have the effect of removing a portion of the skin to be treated (epidermis and possibly surface of the epidermis) by chemical methods, such as the application of compositions comprising high concentrations of agents which stimulate the desquamation of the skin, such as hydroxy acids, for example glycolic acid or salicylic acid, or also other active principles, such as, for example, retinoic acid, resorcinol, trichloroacetic acid or phenol. Thus, the document U.S. Pat. No. 6,787,148 describes anhydrous compositions comprising a phenol and a polyethylene glycol derivative.

Furthermore, hydroxy acids can also be used for cleaning the skin and in particular for cleaning deep into the skin, with possibly a scrubbing effect.

Increasing concentrations of active principle and in particular of hydroxy acids make it possible to increase the effectiveness of the products. However, such concentrations result in serious inconveniences on application and after application (red blotches, smarting, burning feeling). Thus, peels comprising salicylic acid can give rise to cases of salicylism in the event of overdosing or of prolonged application.

For this reason, a search is underway to use derivatives of these acids, in particular acylated derivatives of salicylic acid, such as capryloyl-salicylic acid, as they are generally better tolerated than salicylic acid. However, these salicylic acid derivatives exhibit the disadvantage of being in a crystalline form and of being insoluble or very sparingly soluble in water or in the fatty substances conventionally used in cosmetics. It is therefore difficult to formulate them at a high concentration except for dissolving them in an aqueous/alcoholic solution rich in primary C₂-C₆ alcohols, such as ethanol; however, these primary alcohols are capable of desiccating or irritating the skin. The term “high concentration” is understood here to mean a concentration of at least 5% by weight, with respect to the total weight of the composition.

The need thus remains for cosmetically acceptable and well tolerated aqueous compositions which are capable of dissolving acylated derivatives of salicylic acid at high concentrations.

The Applicant company has found, surprisingly, that, by combining specific alkyl betaines with these salicylic acid derivatives in specific proportions, it is possible to solve the problem at the basis of the invention and to obtain a cosmetically pleasant and well tolerated composition capable of comprising high proportions of salicylic acid derivatives.

A subject-matter of the invention is thus an aqueous composition for topical application comprising at least one (C₈-C₁₄) alkyl betaine and at least one salicylic acid derivative of following formula (I):

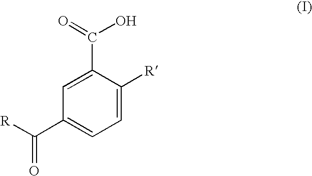

in which:
the R radical denotes a saturated, linear, branched or cyclic, aliphatic chain having from 2 to 22 carbon atoms; an unsaturated chain having from 2 to 22 carbon atoms comprising one or more double bonds which can be conjugated; an aromatic nucleus bonded to the carbonyl radical directly or via saturated or unsaturated aliphatic chains having from 2 to 7 carbon atoms; it being possible for the said groups to be substituted by one or more identical or different substituents chosen from (a) halogen atoms, (b) the trifluoromethyl group, (c) hydroxyl groups in the free form or in the form esterified by an acid having from 1 to 6 carbon atoms or (d) a carboxyl functional group in the free form or in the form esterified by a lower alcohol having from 1 to 6 carbon atoms;
R′ is a hydroxyl group;
and their salts resulting from an inorganic or organic base,
   the molar ratio of the (C₈-C₁₄)alkyl betaine(s) to the salicylic acid derivative(s) being equal to or greater than 1.

The composition of the invention can be used in particular for the peeling or scrubbing of human skin while being well tolerated and while not exhibiting a phenomenon of crystallization of the salicylic acid derivative.

Another subject-matter of the invention is a method for the cosmetic treatment of irregular visible and/or tactile features of the skin, comprising the stages consisting in:

(a) topically applying, to the skin, a composition as defined above,
(b) leaving the composition in contact with the skin for a time sufficient for the composition to act, and
(c) optionally removing the composition by rinsing.

The composition is left in contact with the skin for an application time which has to be sufficient for the composition to act. This time will vary according to the concentration of salicylic acid derivative in the composition and the effect desired. By way of indication, the composition can remain in contact with the skin for a time of at least 5 minutes, generally of between 5 minutes and 12 hours, preferably between 5 minutes and 6 hours, preferentially between 5 minutes and 30 minutes. The composition may or may not be removed at the end of this contact time. Application can be daily or twice daily, or weekly, and repeated over periods of 2 weeks to 6 months; it being possible for this period to be extended or renewed without difficulty.

The composition can also be used for cleaning deep into the skin and for treating greasy skin.

Another subject-matter of the invention is a cosmetic method for cleaning the skin, comprising the stages consisting in:

(a) topically applying, to the skin, a composition as defined above,
(b) massaging the composition over the skin for a time sufficient for the composition to act, and
(c) optionally removing the composition by rinsing.

A further subject-matter of the invention is a method for the cosmetic treatment of greasy skin, comprising the stages consisting in:

(a) topically applying, to the skin, a composition as defined above,
(b) leaving the composition in contact with the skin for a time sufficient for the composition to act, and
(c) optionally removing the composition by rinsing.

As the composition is intended for topical application, it comprises a physiologically acceptable medium. The term “physiologically acceptable medium” is understood to mean a medium compatible with keratinous substances, such as the skin, lips, nails, scalp and/or hair. The composition is in particular a cosmetic or dermatological composition, more particularly intended for peeling and/or cleaning the skin.

The composition is aqueous, that is to say that it comprises water, the amount of water preferably being at least 20% by weight, better still at least 30% by weight and even better still at least 40% by weight, with respect to the total weight of the composition. The amount of water can range, for example, from 20 to 95% by weight, preferably from 30 to 90% by weight and better still from 35 to 80% by weight, with respect to the total weight of the composition. Furthermore, the composition can additionally comprise at least one polyol comprising from 1 to 3 carbon atoms, such as glycerol, propylene glycol, butylene glycol, dipropylene glycol or isopropylene glycol, and their mixtures. The amount of polyol(s) can range, for example, from 0.1 to 20% by weight, better still from 1 to 10% by weight and even better still from 1 to 5% by weight, with respect to the total weight of the composition.

The composition has a pH which can range, for example, from 2 to 10, preferably from 2 to 9 and better still from 3 to 8. According to a preferred embodiment of the invention, the composition has a pH equal to or less than 7, preferably from 3 to 7.

### Salicylic Acid Derivative

As is shown by formula (I), these derivatives are acids (or optionally the salts of these acids) and they differ from salicylic acid through the presence of an acyl group on the aromatic nucleus.

Preferably, the R radical denotes a saturated, linear, branched or cyclic, aliphatic chain comprising from 3 to 11 carbon atoms; an unsaturated chain comprising from 3 to 17 carbon atoms and comprising one or more conjugated or nonconjugated double bonds; it being possible for the said hydrocarbon chains to be substituted by one or more identical or different substituents chosen from (a) halogen atoms, (b) the trifluoromethyl group, (c) hydroxyl groups in the free form or in the form esterified by an acid having from 1 to 6 carbon atoms or (d) a carboxyl functional group in the free form or in the form esterified by a lower alcohol having from 1 to 6 carbon atoms;
and their salts obtained by salification by an inorganic or organic base.

The compounds which are more particularly preferred are those in which the R radical is a C₃-C₁₁ alkyl group.

Mention may be made, among the compounds of formula (I) which are particularly preferred, of: 5-(n-octanoyl)salicylic acid (or capryloylsalicylic acid); 5-(n-decanoyl)salicylic acid; 5-(n-dodecanoyl)salicylic acid; 5-(n-heptyloxy)salicylic acid; their corresponding salts, and their mixtures.

Use will more particularly be made of 5-(n-octanoyl)salicylic acid (molecular weight 264 g/mol).

The salts of the compounds of formula (I) can be obtained by salification by an inorganic or organic base. Mention may be made, as examples of inorganic base, of alkali metal or alkaline earth metal hydroxides, such as sodium hydroxide or potassium hydroxide, or aqueous ammonia.

Mention may be made, among organic bases, of amines and alkanolamines. Quaternary salts, such as those described in document FR-A-2 607 498, are particularly advantageous.

The compounds of formula (I) which can be used according to the invention are described in the documents U.S. Pat. No. 6,159,479, U.S. Pat. No. 5,558,871, FR-A-2 581 542, FR-A-2 607 498, U.S. Pat. No. 4,767,750, EP-A-378 936, U.S. Pat. No. 5,267,407, U.S. Pat. No. 5,667,789, U.S. Pat. No. 5,580,549 and EP-A-570 230.

The amount of salicylic acid derivative(s) of formula (I) (as active material) depends on the desired objective and on the type of skin. It can range, for example, from 2 to 20% by weight and preferably from 5 to 10% by weight, with respect to the total weight of the composition.

### (C₈-C₁₄)Alkyl Betaines

(C₈-C₁₄)Alkyl betaines comprise an alkyl group having from 8 to 14 carbon atoms. Use is preferably made, as (C₈-C₁₄) alkyl betaine, of a (C₁₋₂)alkyl betaine, more specifically lauryl betaine, such as, for example, the product sold under the name Empigen BB LS® by Huntsman. Lauryl betaine has a molecular weight of 278 g/mol.

The amount of (C₈-C₁₄) alkyl betaine(s) (as active material) depends on the desired objective and on the type of skin. It can range, for example, from 2 to 20% by weight and preferably from 5 to 10% by weight, with respect to the total weight of the composition.

The amount of (C₈-C₁₄) alkyl betaine(s) also depends on the amount of salicylic acid derivative(s) of formula (I). The molar ratio of the alkyl betaine(s) to the salicylic acid derivative(s) of formula (I) (as active material) is equal to or greater than 1. It can range, for example, from 1 to 1.5, preferably from 1.1 to 1.5 and better still from 1.1 to 1.3.

### Other Compounds

In addition to the salicylic acid derivative, the composition according to the invention can comprise one or more active principles commonly used in peeling or cleaning compositions, in particular acidic active principles and especially hydroxy acids chosen from α-hydroxy acids, β-hydroxy acids other than the derivatives of formula (I), α-keto acids, β-keto acids, and their mixtures.

Mention may more particularly be made of α-hydroxy acids and, as α-hydroxy acids, citric acid, lactic acid, glycolic acid, tartaric acid, malic acid, mandelic acid, methyllactic acid, glucuronic acid, pyruvic acid, phenyllacetic acid, gluconic acid, galacturonic acid and the plant extracts comprising these acids. Use is preferably made of glycolic acid, lactic acid and the plant extracts comprising them.

These acidic active principles and in particular the hydroxy acids can be present in an amount ranging from 0.1 to 30% by weight, better still from 0.5 to 20% by weight, even better still from 1 to 15% by weight, preferably from 5 to 12% by weight, with respect to the total weight of the composition.

The compositions according to the invention can be provided in any formulation form generally used in the cosmetic and dermatological fields, in particular in the form of aqueous gels, of lotions or of emulsions. These compositions are prepared according to the normal methods. According to a preferred embodiment of the invention, the composition is provided in the form of an aqueous or aqueous/glycolic gel or of an aqueous or aqueous/glycolic solution.

The composition according to the invention can comprise at least one oil and thus an oily phase, in particular for introducing a care effect or introducing better tolerance or also for improving the solubilization of some lipophilic active principles. The oily phase then preferably comprises at least one oil, in particular a physiologically acceptable oil. It can additionally comprise other fatty substances. The amount of oily phase is at most 10% of the total weight of the composition and it can range, for example, from 5 to 10% of the total weight of the composition.

Mention may be made, as oils which can be used in the composition of the invention, for example, of: hydrocarbon oils of animal origin or of vegetable origin, synthetic esters and ethers, linear or branched hydrocarbons of mineral or synthetic origin, such as liquid paraffins, which may or may not be volatile, and their derivatives, petrolatum, polydecenes, hydrogenated polyisobutene, such as Parleam oil; fatty alcohols having from 8 to 26 carbon atoms, volatile or nonvolatile silicone oils comprising a linear or cyclic silicone chain which are liquid or pasty at ambient temperature, and their mixtures.

The term “hydrocarbon oil” is understood to mean, in the list of the oils mentioned above, any oil predominantly comprising carbon and hydrogen atoms and optionally ester, ether, fluorinated, carboxylic acid and/or alcohol groups.

If necessary, the composition can optionally comprise an appropriate emulsifier chosen from those conventionally used in the cosmetics field.

In a known way, the composition of the invention can also comprise adjuvants usual in the cosmetics or dermatological field, such as hydrophilic surfactants, hydrophilic or lipophilic gelling agents, hydrophilic or lipophilic active principles other than those mentioned above, preservatives (for example phenoxy-ethanol and parabens), antioxidants, solvents, fragrances, fillers, bactericides, odour absorbers, colouring materials, or pH adjusters (acid or base or buffer). The amounts of these various adjuvants are those conventionally used in the field under consideration, for example from 0.01 to 20% and better still from 0.01 to 10% of the total weight of the composition.

Of course, a person skilled in the art will take care to choose the optional additive or additives to be added to the composition according to the invention and their amounts so that the advantageous properties intrinsically attached to the composition in accordance with the invention are not, or not substantially, detrimentally affected by the envisaged addition.

The composition can comprise one or more fillers. The composition of the invention can comprise, as fillers, for example, inorganic particles, such as clays, silicas, metal oxides, such as titanium dioxide or zinc oxide, mica, and/or organic fillers, such as polyamide (Nylon) particles and in particular those sold under the Orgasol names by Atochem; polyethylene powders; microspheres based on acrylic copolymers, such as those made of ethylene glycol dimethacrylate/lauryl methacrylate copolymer sold by Dow Corning under the Polytrap name; polymethyl methacrylate microspheres sold under the name Microsphere M-100 by Matsumoto or under the name Covabead LH85 by Wackherr; ethylene/acrylate copolymer powders, such as those sold under the name Flobeads by Sumitomo Seika Chemicals; expanded powders, such as hollow microspheres and in particular the microspheres formed of a terpolymer of vinylidene chloride, of acrylonitrile and of methacrylate sold under the name Expancel by Kemanord Plast; powders formed of natural organic materials, such as starch powders, in particular powders formed of maize, wheat or rice starches which may or may not be crosslinked, such as powders formed of starch crosslinked by octenyl succinic anhydride which are sold under the name Dry-Flo by National Starch; silicone resin microbeads, such as those sold under the name Tospearl by Toshiba Silicone, in particular Tospearl 240; and their mixtures. The amount of filler(s) can range, for example, from 0.05 to 10% by weight and better still from 0.1 to 5% by weight, with respect to the total weight of the composition.

The composition can also comprise any appropriate active principle, such as, for example, urea and its hydroxylated derivatives, such as the N-(2-hydroxy-ethyl)urea sold under the name Hydrovance by National Starch; hyaluronic acid; moisturizing polymers; vitamins, such as vitamins A, C, E, B3, B5, K and their derivatives, in particular their esters; and sequestering agents, such as EDTA.

The composition according to the invention can be prepared by any appropriate means. According to a preferred embodiment, when the content of salicylic acid derivative of formula (I) is high, the composition is prepared by heating the alkyl betaine in aqueous solution (for example comprising 30% of active material, as is the case for Empigen BB LS) to approximately 60° C. with shearing and then gradual incorporation of the salicylic acid derivative of formula (I) and subsequently of the water heated to approximately 60° C. The mixture is then heated at 80° C. approximately for approximately 10 minutes and cooled to ambient temperature (20 to 25° C.)

The composition according to the invention can be applied by any means which makes possible uniform distribution over the skin, in particular using a cotton wool swab, a rod, a brush, a gauze, a spatula or a pad or also by spraying, and it may or may not be removed. It can be removed, for example, by rinsing with water or simple wiping.

As indicated above, the composition according to the invention can be employed in a peeling method targeted at toning down irregular visible and/or tactile features of the skin and in particular at toning down wrinkles and fine lines and/or pigment blemishes and/or scars, in particular marks of acne, and/or at unblocking the pores of the skin and giving greater radiance to the skin. The composition can thus be applied in particular to the face and/or the neck and/or the shoulders and/or the hands and/or the back.

In order to optimize its effects, the peeling method according to the invention can comprise one or more additional stages, for example a preliminary stage of preparing the skin for peeling using compositions including smaller amounts of active principles than the peeling composition described above, or an additional stage of caring for the skin after peeling.

The use of the above stage of preparing the skin for peeling additionally makes it possible to detect possible allergy to active principles and to improve the effectiveness and the homogeneity of the peeling.

The method according to the invention, including the optional preliminary and additional stages, can be carried out just once or renewed up to 5 times, if necessary, the peeling sessions preferably taking place every 1 to 8 weeks.

For the cleaning or treating of greasy skin, the composition can also be applied in particular to the face and/or the neck and/or the shoulders and/or the hands and/or the back.

The invention will now be illustrated by the following nonlimiting examples. In these examples, the amounts are shown as percentage by weight of active material. The compounds are by INCI name or by chemical name as the case may be.

### EXAMPLES 1 TO 4

### Peeling Compositions

| Composition | Example 1 | Example 2 | Example 3 | Example 4 |
|---|---|---|---|---|
| 5-(n-Octanoyl) | 10% | 10% | 10% | 10% |
| salicylic | | | | |
| acid | | | | |
| Lauryl betaine (1) | 12% | 15% | 12% | 12% |
| Glycolic acid (2) | — | 15% | — | — |
| Pure sodium | — | — | 1.42 | 1.475 |
| hydroxide | | | | |
| Water | q.s. for | q.s. for | q.s. for | q.s. for |
| | 100% | 100% | 100% | 100% |
| R* | 1.139 | 1.424 | 1.139 | 1.139 |
| pH | 3 | 2 | 7 | 10 |
| Appearance | Viscous | Viscous | Fluid | Transparent |
| | gel. | gel. | translucent | solution. |
| | No crystals | No | gel. | No crystals |
| | | crystals | No crystals | |

(1) Empigen BB/LS (Huntsman) comprising 30% of active material (i.e., 40% of starting material in the examples)
(2) Glypure 99 (Dupont) comprising 100% of active material (i.e., 30% of starting material in the example)
*R = molar ratio of the lauryl betaine to the 5-(n-octanoyl)salicylic acid

The compositions obtained remained stable without crystals for at least two months between 4 and 45° C.

### COMPARATIVE EXAMPLES 1 to 4

| | Comparative | Comparative | Comparative | Comparative |
|---|---|---|---|---|
| Composition | Example 1 | Example 2 | Example 3 | Example 4 |
| Myristyldimethyl- | 12% | | | |
| propyl | | | | |
| sulphobetaine (3) | | | | |
| Cetyl betaine (4) | | 12% | | |
| Cocamidopropyl | | | 12% | |
| betaine (5) | | | | |
| Cocoylamidopropyl | | | | 12% |
| dimethyl hydroxy- | | | | |
| propyl | | | | |
| sulphobetaine (6) | | | | |
| 5-(n- | 10% | 5% | 5% | 5% |
| Octanoyl)salicylic | | | | |
| acid | | | | |
| Water | q.s. for | q.s. for | q.s. for | q.s. for |
| | 100% | 100% | 100% | 100% |
| Appearance | presence of | presence of | phase | presence of crystals |
| | crystals | crystals | separation + | |
| | | | presence of crystals | |

(3) Ralufon DM (Raschig) comprising 98% of active material (i.e., 12.25% of starting material in the example)
(4) Detaine PB (Deforest Entreprises) comprising 50% of active material (i.e., 24% of starting material in the example)
(5) Mirataine BET/CT (Rhodia) comprising 30.5% of active material (i.e., 39.345% of starting material in the example)
(6) Rewoteric AM CAS (Degussa) comprising 50% of active material (i.e., 24% of starting material in the example)

The compositions of the comparative examples described above crystallized in less than one week. Thus, the comparative examples show that betaine derivatives other than those claimed do not make it possible to obtain the desired result.

### COMPARATIVE EXAMPLE 5

| Composition | Comparative Example 5 |
|---|---|
| 5-(n-Octanoyl)salicylic acid | 10% |
| Lauryl betaine (1) | 30% |
| Water | q.s. for 100% |
| R* | 0.85 |
| pH | 3.3 |
| Appearance | Significant crystals |
| | from the moment of |
| | the mixing |

Comparative Example 5 shows that the objective of the invention is not achieved when the molar ratio of lauryl betaine to 5-(n-octanoyl)salicylic acid is less than 1.

### Test: Improvement in the Tolerance and in the Effectiveness

### 1) In Vitro Tolerance of the Peeling Products

The tolerance of the products is evaluated in vitro on reconstructed epidermi (EpiSkin®). The protocol used (EpiPeel) makes it possible to deploy a strategy of evaluation of highly concentrated active principles in a simplex formulation. This protocol uses the complementary nature of two tests carried out in vitro on reconstructed epidermi: test predictive of corrosion (validated in Europe as replacement test for the animal model, TG 431) and test predictive of the irritant potential of chemical products. EpiPeel makes it possible to define a “standard” gradation of the effects over five levels of activity. Its current orientation is targeted at the evaluation of the cutaneous tolerance of products formulated or not formulated at high concentrations, the recommended contact times of which are of short duration and carried out under control.

### EpiPeel Predictive Model

| | Level 1 | Level 2 | Level 3 | Level 4 | Level 5 |
|---|---|---|---|---|---|
| Corrosion EU | R35 | R34 | R34 | NC | NC |
| Corrosion UN | Class I | Class II | Class III | NC | NC |
| Tolerance | | | | Irritating | Nonirritating |

NC: noncorrosive
UN: United Nations Classification
EU: European Union Classification

Model predictive of corrosion (OECD Guideline TG 431):

R35/class I: very corrosive
% viability<35% after 3 min of contact
   R34/class II: corrosive
% viability≧35% after 3 min of contact and <35% after 1 hour of contact
   R34/class III: corrosive
% viability≧35% after 1 hour of contact and <35% after 4 hours of contact

This test showed that the corrosive potential of Example 1 (categorized Level 4) is lower than that of a 10% solution of 5-(n-octanoyl)salicylic acid in ethanol or a 10% solution of 5-(n-octanoyl)salicylic acid in an aqueous/alcoholic solution comprising 30% water, 50% ethanol and 20% propylene glycol (categorized Level 2). Example 4 is positioned with the same test as noncorrosive and nonirritating (Level 5).

### 2) Test of Peeling Effectiveness In Vitro

An in vitro test carried out in order to determine the effectiveness of the peeling products (counting of the number of corneocytes released after application of the composition) showed that Example 1 according to the invention is more effective than a 10% solution of 5-(n-octanoyl)salicylic acid in ethanol.

1. An aqueous composition for topical application comprising:
at least one (C₈-C₁₄)alkyl betaine and
at least one salicylic acid derivative of following formula (I): 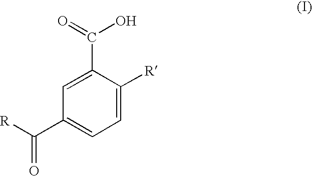
wherein:
the R radical denotes a saturated, linear, branched or cyclic, aliphatic chain having from 2 to 22 carbon atoms; an unsaturated chain having from 2 to 22 carbon atoms comprising one or more double bonds which can be conjugated; an aromatic nucleus bonded to the carbonyl radical directly or via saturated or unsaturated aliphatic chains having from 2 to 7 carbon atoms; it being possible for the said groups to be substituted by one or more identical or different substituents chosen from (a) halogen atoms, (b) the trifluoromethyl group, (c) hydroxyl groups in the free form or in the form esterified by an acid having from 1 to 6 carbon atoms or (d) a carboxyl functional group in the free form or in the form esterified by a lower alcohol having from 1 to 6 carbon atoms;
R′ is a hydroxyl group;
and their salts resulting from an inorganic or organic base,
the molar ratio of the (C₈-C₁₄)alkyl betaine(s) to the salicylic acid derivative(s) being equal to or greater than 1.

2. The composition according to claim 1, further comprising at least 20% by weight of water, with respect to the total weight of the composition.

3. The composition according to claim 1, wherein in the formula (I), the R radical denotes a saturated, linear, branched or cyclic, aliphatic chain comprising from 3 to 11 carbon atoms; an unsaturated chain comprising from 3 to 17 carbon atoms and comprising one or more conjugated or nonconjugated double bonds; it being possible for the said hydrocarbon chains to be substituted by one or more identical or different substituents chosen from (a) halogen atoms, (b) the trifluoromethyl group, (c) hydroxyl groups in the free form or in the form esterified by an acid having from 1 to 6 carbon atoms or (d) a carboxyl functional group in the free form or in the form esterified by a lower alcohol having from 1 to 6 carbon atoms;
and their salts obtained by salification by an inorganic or organic base.

4. The composition according to claim 1, wherein the salicylic acid derivative is selected from the group of derivatives consisting of 5-(n-octanoyl)salicylic acid, 5-(n-decanoyl)salicylic acid, 5-(n-dodecanoyl)salicylic acid, 5-(n-heptyloxy)salicylic acid, a corresponding salt thereof, and a mixture thereof.

5. The composition according to claim 1, wherein an amount of salicylic acid derivative(s) ranges from 2 to 20% by weight with respect to the total weight of the composition.

6. The composition according to claim 1, wherein the (C₈-C₁₄)alkyl betaine is lauryl betaine.

7. The composition according to claim 1, wherein an amount of (C₈-C₁₄)alkyl betaine(s) ranges from 2 to 20% by weight with respect to the total weight of the composition.

8. The composition according to claim 1, wherein a molar ratio of the alkyl betaine(s) to the salicylic acid derivative(s) ranges from 1.1 to 1.5.

9. The composition according to claim 1, additionally comprising one or more hydroxy acids selected from the group consisting of α-hydroxy acids, β-hydroxy acids other than the derivatives of formula (I), α-keto acids and β-keto acids.

10. The composition according to claim 9, wherein the one or more α-hydroxy acids are selected from the group consisting of glycolic acid, lactic acid and plant extracts comprising them.

11. The composition according to claim 9 wherein an amount of the one or more hydroxy acids ranges from 0.1 to 30% by weight with respect to the total weight of the composition.

12. The composition according to claim 1, wherein a pH is in the range of from 2 to 10.

13. A method for the cosmetic treatment of irregular visible and/or tactile features of the skin, comprising:
(a) topically applying, to the skin, a composition according to claims 1;
(b) leaving the composition in contact with the skin for a time sufficient for the composition to act, and
(c) optionally removing the composition by rinsing.

14. The method according to claim 13, wherein the composition according to claim 1 is left in contact with the skin for a time of between 5 minutes and 12 hours.

15. A cosmetic method for cleaning the skin, comprising:
(a) topically applying, to the skin, a composition according to claims 1;
(b) massaging the composition in contact with the skin for a time sufficient for the composition to act, and
(c) optionally removing the composition by rinsing.

16. A method for the cosmetic treatment of greasy skin, comprising:
(a) topically applying, to the skin, a composition according to claims 1;
(b) leaving the composition in contact with the skin for a time sufficient for the composition to act, and
(c) optionally removing the composition by rinsing.
